(19) United States
(12) Patent Application Publication
(10) Pub. No.: US 2007/0142456 A1
(43) Pub. Date: Jun. 21, 2007

(54) Method for inhibiting cancer development by fatty acid synthase inhibitors

(75) Inventors: Francis P. Kuhajda, Baltimore, MD (US); Elizabeth M. Jaffee, Baltimore, MD (US); Craig A. Townsend, Baltimore, MD (US)

Correspondence Address: COVINGTON & BURLING, LLP;ATTN: PATENT DOCKETING, 1201 PENNSYLVANIA AVENUE, N.W., WASHINGTON, DC, 20004-2401, US

(73) Assignees: FASgen, LLC, Baltimore, MD (US); John Hopkins University, Baltimore, MD (US)

(21) Appl. No.: 10/533,311

(22) PCT Filed: Oct. 31, 2003
(86) PCT No.: PCT/US03/34658

§ 371 Date: Apr. 7, 2006

Related U.S. Application Data
(60) Provisional application No. 60/422,746, filed on Oct. 31, 2002.

(51) Int. Cl. A61K 31/381; A61K 31/365

(52) U.S. Cl. 514/446; 514/473

(57) ABSTRACT

A method for inhibiting or preventing cancer development by the administration of fatty acid synthase (FAS) inhibitors. In particular, the present invention prohibits or delays the development of invasive cancer from pre-malignant (non-invasive) lesions that express FAS. Compositions containing FAS inhibitors also are provided, as well as methods for administering the FAS inhibitors and compositions to patients in need thereof.

### FIELD OF THE INVENTION

The present invention relates to a method for inhibiting or preventing cancer development by the administration of fatty acid synthase (FAS) inhibitors. In particular, the present invention prohibits or delays the development of invasive cancer from pre-malignant (non-invasive) lesions that express FAS. Compositions containing FAS inhibitors also are provided, as well as methods for administering the FAS inhibitors and compositions to patients in need thereof.

### BACKGROUND OF THE INVENTION

Fatty acids have three primary roles in the physiology of cells. First, they are the building bocks of biological membranes. Second, fatty acid derivatives serve as hormones and intracellular messengers. Third, fatty acids are fuel molecules that can be stored in adipose tissue as triacylglycerols, which are also known as neutral fats.

There are four primary enzymes involved in the fatty acid synthetic pathway, fatty acid synthase (FAS), acetyl CoA carboxylase (ACC), malic enzyme, and citric lyase. The principal enzyme is FAS, which catalyzes the NADPH-dependent condensation of the precursors malonyl-CoA and acetyl-CoA to produce fatty acids. NADPH is a reducing agent that serves as an essential electron donor in the two reductase steps (enoyl reductase and β-ketoacyl reductase) in fatty acid synthase. The other three enzymes (i.e., ACC, malic enzyme, and citric lyase) produce the necessary precursors. Other enzymes, such as, for example, the enzymes that produce NADPH, are also involved in fatty acid synthesis.

FAS has an Enzyme Commission (E.C.) No. 2.3.1.85 and is also known as fatty acid synthetase, fatty acid ligase, as well as its systematic name acyl-CoA:malonyl-CoA C-acyltransferase (decarboxylating, oxoacyl- and enoyl-reducing and thioester-hydrolysing). There are seven distinct enzymes involved in the FAS catalyzed synthesis of fatty acids: acetyl transacylase, malonyl transacylase, beta-ketoacyl synthetase (condensing enzyme), beta-ketoacyl reductase, beta-hydroxyacyl dehydrase, enoyl reductase, and thioesterase (Wakil, S., “Fatty acid synthase, a proficient multifunctional enzyme.” Biochemistry, 28: 4523-4530, 1989). All seven of these enzymes together comprise FAS.

Of the four enzymes in the fatty acid synthetic pathway, FAS is the preferred target for inhibition because it acts only within the fatty acid synthetic pathway, while the other three enzymes are implicated in other cellular functions. Therefore, inhibition of one of the other three enzymes is more likely to affect normal cells.

FAS inhibitors can be identified by the ability of a compound to inhibit the enzymatic activity of purified FAS. FAS activity can be assayed by numerous means known in the art, such as, for example, measuring the oxidation of NADPH in the presence of malonyl CoA (Dils, R. and Carey, E. M., “Fatty acid synthase from rabbit mammary gland,” Methods Enzymol, 35: 74-83, 1975). Other information relating to determination of whether a compound is an FAS inhibitor may be found in U.S. Pat. No. 5,981,575, the disclosure of which is hereby incorporated by reference.

Of the seven enzymatic steps carried out by FAS, the step catalyzed by the condensing enzyme (i.e., beta-ketoacyl synthetase) is the preferred candidate for inhibitors that reduce or stop fatty acid synthesis. The condensing enzyme of the FAS complex is well characterized in terms of structure and function. The active center of the condensing enzyme contains a critical cysteine thiol, which is the target of antilipidemic reagents, such as, for example, the inhibitor 2,3-epoxy-4-oxo-7,10-dodecadienoylamide (hereinafter “cerulenin”).

Preferred inhibitors of the condensing enzyme include a wide range of chemical compounds, including alkylating agents, oxidants, and reagents capable of undergoing disulphide interchange. Confirmation of the inhibitory activity of such compounds may be demonstrated by observing the effect of the compound on assays measuring their effect on the activity of purified human fatty acid synthase, or on the incorporation of [¹⁴C]acetate into total lipids. (Pizer, E. S., Thupari, J., Han, W. F., Pinn, M. L., Chrest, F. J., Frehywot, G. L., Townsend, C. A., and Kuhajda, F. P., “Malonyl-coenzyme-A is a potential mediator of cytotoxicity induced by fatty acid synthase inhibition in human breast cancer cells and xenografts,” Cancer Research, 60: 213-218, 2000). Cerulenin is an example of such an inhibitor. Cerulenin covalently binds to the critical cysteine thiol group in the active site of the condensing enzyme of FAS, inactivating this key enzymatic step (Funabashi, H., Kawaguchi, A., Tomoda, H., Omura, S., Okuda, S., and Iwasaki, S. Binding site of cerulenin in fatty acid synthetase. J. Biochem., 105: 751-755, 1989).

Various other compounds have been shown to inhibit FAS. Table 1, set forth below, lists several known FAS inhibitors. Preferably, inhibitors according to this invention will exhibit a suitable therapeutic index, safety profile, as well as efficacy, by showing IC₅₀ for FAS inhibition that is lower than the LD₅₀; more preferably LD₅₀ is at least an order of magnitude higher than IC₅₀.

TABLE 1
Representative Inhibitors Of The Enzymes Of The Fatty
Acid Synthesis Pathway
Inhibitors of Fatty Acid Synthase

1,3-dibromopropanone
Ellman's reagent (5,5′-dithiobis(2-nitrobenzoic acid), DTNB)
4-(4′-chlorobenzyloxy)benzyl nicotinate (KCD-232)
4-(4′-chlorobenzyloxy)benzoic acid (MII)
2(5(4-chlorophenyl)pentyl)oxirane-2-carboxylate (POCA) and its
CoA derivative
ethoxyformic anhydride
cerulenin
phenyocerulenin
melarsoprol
iodoacetate
phenylarsineoxide
pentostam
melittin
thiolactomycin

FAS inhibitors have been disclosed as agents for inducing weight loss and for inhibiting the growth of pre-existing cancer cells. For example, U.S. Pat. No. 5,981,575 (“the '575 patent”) discloses a method for inducing weight loss by the administration of a class of FAS inhibitors (γ-substituted-α-methylene-β-carboxy-γ-butyrolactone compounds). The '575 patent also discloses that these compounds are useful for inhibiting the growth of pre-existing cancer cells. U.S. Pat. No. 5,759,837 (“the '837 patent”), discloses a method for treating pre-existing cancer by administering an FAS inhibitor at an amount that is selectively cytotoxic to cancer cells, but not to other types of non-transformed (normal) cells. However, neither the '575 patent nor the '837 patent disclose the administration of these compounds prior to cancer development (i.e., prior to the initial appearance of cancerous cells), much less any method involving pre-cancerous lesions.

Numerous technologies have recently been developed that detect pre-cancerous states in patients, allowing treatment to begin even before the initial appearance of cancerous cells. Such early diagnosis allows preventive treatment to begin that substantially reduces the risk of cancer development. Known techniques for early screening include, for example, using optically, sonographic, or radiologically guided needle biopsy, fine needle aspiration, and exfoliative cytology to detect pre-cancerous lesions in various tissue types, such as, for example, the breast, aerodigestive tract, pancreas, prostate, and colon.

Improvements in cancer morbidity and cancer survival statistics are primarily based upon the early detection of the disease when the tumor size is small and the cancer is confined to the site of origin. The slight decrease in the mortality rate for breast cancer in the last 2 years is likely due in part to early detection (Ahmedin, J., Thomas, A., Murray, T., and Thun, M., “Cancer Statistics 2002,” CA Cancer J Clin, 52: 23-47, 2002). However, despite the recent advances in early diagnosis, the mortality rate for many cancers has not shown concomitant improvement. A further potentially very significant improvement in cancer morbidity and mortality would follow from an effective treatment of pre-malignant lesions that would prevent or delay the development of invasive cancers.

The present invention compliments the recent advances in early diagnosis by providing a method for treating the pre-cancerous state in a subject (i.e., inhibiting cancer development) by the administration of an FAS inhibitor.

### SUMMARY OF THE INVENTION

The present invention provides a method for inhibiting cancer development by the administration of FAS inhibitors. The method of the present invention is particularly useful in delaying or preventing breast cancer development from pre-malignant lesions that express FAS. Compositions containing the FAS inhibitors also are provided, as well as methods for administering the FAS inhibitors and compositions to patients in need thereof.

Accordingly, in one embodiment, the present invention provides a method of inhibiting cancer development involving the administration to a subject in need thereof of an effective amount of an FAS inhibitor.

In another embodiment, the present invention provides cancer development inhibiting pharmaceutical compositions containing pharmaceutically acceptable additives and effective cancer development inhibiting amounts of an FAS inhibitor.

### BRIEF DESCRIPTION OF THE DRAWINGS

FIG. 1 illustrates the inhibition of fatty acid synthesis by cerulenin and tetrahydro-3-methylene-2-oxo-5-n-octyl-4-furancarboxylic acid (hereinafter “C75”) in NT5 cancer cells.

FIG. 2 illustrates that FAS inhibitors can inhibit NT5 cancer cell growth in vitro.

FIG. 3 illustrates that FAS inhibitors can reduce the growth of NT5 cancer cell allografts in mice.

FIG. 4 illustrates that FAS inhibitors can inhibit cancer development in the HER-2/neu breast cancer transgenic mouse model.

### DETAILED DESCRIPTION OF THE INVENTION

The present invention provides a method for inhibiting cancer development by the administration of FAS inhibitors. In particular, the present invention provides a method of inhibiting cancer development involving the administration to a subject in need thereof an effective amount of an FAS inhibitor.

The present invention also provides a composition containing an FAS inhibitor useful for inhibiting cancer development. In particular, the present invention provides a cancer development inhibiting pharmaceutical composition containing a pharmaceutically acceptable additive and an effective cancer development inhibiting amount of an FAS inhibitor.

As used herein, the term “inhibiting” is understood to mean preventing, suppressing, retarding, blocking or delaying cancer development, such as, for example, by stimulating, inducing or triggering apoptosis (i.e., genetically determined cell death) in pre-cancerous cells.

As used herein, the term “cancer development” is understood to mean the initial appearance of cancerous cells. By “cancerous cells,” we mean cells which have the property of autonomous proliferation and have invaded adjacent tissues.

As used herein, the term “administration” is understood to mean any of a multitude of possible means of administration commonly used in the art, such as, for example, orally, rectally, nasally, or parenterally, and the like, wherein parenteral administration includes, for example, intravenous, intramuscular, intraperitoneal, intrapleural, intravesicular, intrathecal, subcutaneous, as well as topical administration. In addition, “administration” includes administration via any of a multitude of pharmaceutical composition forms commonly used in the art.

Preferred pharmaceutical compositions include oral compositions, such as, for example, solid forms (e.g., tablets, capsules, powders, pills, or granules) or liquid forms (e.g., syrups, emulsions or suspensions); rectal compositions, such as, for example, suppositories; and parenteral compositions, such as, for example, compositions suitable for injection or infusion.

As used herein, the term “subject in need thereof” is understood to include subjects who have been diagnosed as pre-cancerous, or who may have a predisposition to develop the disease, genetic or otherwise. In a preferred mode, this invention is not directed to treatment of subjects who are taking FAS inhibitors for some purpose other than to treat the pre-cancerous condition, such as, for example, for weight loss.

Preferably the subject has not developed cancer of the type for which treatment is sought. In addition, the subject may have one or more pre-cancerous lesions. The pre-cancerous lesions may preferably express FAS, or both FAS and the neu protein. Although the pre-cancerous lesions may occur n any tissue, this invention particularly provides therapy for lesions in the breast, oral cavity, lung, bile duct, stomach, prostate, or any combination thereof that express FAS. Preferably the subject is a mammal, more preferably a human.

As used herein, the term “effective cancer development inhibiting amount” is understood to mean an amount of FAS inhibitor necessary to achieve the desired result of inhibiting cancer development. It is also understood that the effective amount will normally be determined by a prescribing physician and that the amount will vary according to the age, weight and response of the individual subject, as well as the severity of the subject's symptoms (if the patient has symptoms from the pre-cancerous lesion) and the potency of the particular compound being administered. Preferably, the effective amount is in the range from about 60 mg/kg to about 7.5 mg/kg per week, more preferably in the range from about 30 mg/kg to about 7.5 mg/kg per week, most preferably in the range from about 15 mg/kg to about 7.5 mg/kg per week. The effective amount may be administered in single or divided doses.

As used herein, the term “FAS inhibitor” is understood to mean a compound which directly inhibits the FAS enzyme. Direct inhibition means that the inhibitor reduces FAS activity by direct action on the enzyme rather than as a secondary consequence of some other action of the compound, such as, for example, a reduction in all cellular activities. FAS inhibition can be determined by the means set forth in U.S. Pat. No. 5,981,575.

Preferably, the FAS inhibitor is one of the following compounds: C75 (i.e., tetrahydro-3-methylene-2-oxo-5-n-octyl-4-furancarboxylic acid); cerulenin (i.e., 2,3-epoxy-4-oxo-7,10-dodecadienoylamide); 1,3-dibromopropanone; Ellman's reagent (5,5′-dithiobis(2-nitrobenzoic acid), DTNB); 4-(4′-chlorobenzyloxy)benzyl nicotinate (KCD-232); 4-(4′-chlorobenzyloxy)benzoic acid (MII); 2(5(4-chlorophenyl)pentyl)oxirane-2-carboxylate (POCA) and its CoA derivative; ethoxyformic anhydride; thiolactomycin; phenyocerulenin; melarsoprol; iodoacetate; phenylarsineoxide; pentostam; melittin; or methyl malonyl CoA. One preferred FAS inhibitor is C75. Other preferred FAS compounds are those disclosed in U.S. Patent Application No. 60/394,585 (the disclosure of which is hereby incorporated by reference): 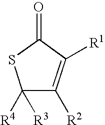
wherein:
R¹═H, C₁-C₂₀ alkyl, cycloalkyl, alkenyl, aryl, arylalkyl, or alkylaryl, —CH₂OR⁵, —C(O)R⁵, —CO(O)R⁵, —C(O)NR⁵R⁶, —CH₂C(O)R⁵, or —CH₂C(O)NHR⁵, where R⁵ and R⁶ are each independently H, C₁-C₁₀ alkyl, cycloalkyl, alkenyl, aryl, arylalkyl, or alkylaryl, optionally containing one or more halogen atoms.
R²═—OH, —OR⁷, —OCH₂C(O)R⁷, —OCH₂C(O)NHR⁷, —OC(O)R⁷, —OC(O)OR⁷, —OC(O)NR⁷R⁸, where R⁷ and R⁸ are each independently H, C₁-C₂₀ alkyl, cycloalkyl, alkenyl, aryl, arylalkyl, or alkylaryl, and where R⁷ and R⁸ can each optionally contain halogen atoms;
R³ and R⁴, the same or different from each other, are C₁-C₂₀ alkyl, cycloalkyl, alkenyl, aryl, arylalkyl, or alkylaryl.

Another group of preferred FAS-inhibitors are those disclosed in U.S. Patent Application Ser. No. 60/392,809 (the disclosure of which is hereby incorporated by reference): 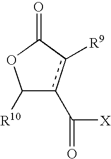
R⁹═H, or C₁-C₂₀ alkyl, cycloalkyl, alkenyl, aryl, arylalkyl, or alkylaryl, ═CHR¹¹, —C(O)OR¹¹, —C(O)R¹¹, —CH₂C(O)OR¹¹, —CH₂C(O)NHR¹¹, where R¹¹ is H or C₁-C₁₀ alkyl, cycloalkyl, or alkenyl;
R¹⁰═C₁-C₂₀ alkyl, cycloalkyl, alkenyl, aryl, arylalkyl, or alkylaryl;
X═—OR¹², or —NHR¹², where R¹² is H, C₁-C₂₀ alkyl, cycloalkyl, alkenyl, aryl, arylalkyl, or alkylaryl, the R¹² group optionally containing a carbonyl group, a carboxyl group, a carboxyamide group, an alcohol group, or an ether group, the R¹² group further optionally containing one or more halogen atoms;
with the proviso that when R⁹ is ═CH₂, then X is not —OH.

As used herein, the term “additive” is understood to mean any of a multitude of possible additives commonly used in the art, such as, for example, carriers, excipients, diluting agents, fillers, or combinations thereof. Preferred examples of additives are water, alcohols, gelatin, saccharose, pectin, magnesium stearate, stearic acid, talc, various oils of animal or plant origin, glycols, starch and starch derivatives, silica, lactose, lactose monohydrate, cellulose and cellulose derivatives, magnesium stearate, calcium stearate, calcium hydrogen phosphate, PVP or povidone, mannitol, sorbitol, gelatin, sugar alcohols, stearic acid, acryl derivatives, alginic acid, .alpha.-octadecyl-.OMEGA.-hydroxypoly-(oxyethylen)-5-sorbic acid-H₂O, gum arabic, flavoring substances, ascorbic acid, calcium carbonate, calcium hydrogen phosphate, calcium phosphate, calcium stearate, carmellose sodium, cellulose, cellulose derivatives, dimethicon, coloring agents, gelatin, glucose syrup, highly dispersed silica, potassium benzoate, lactose monohydrate, Macrogol, magnesium carbonate, magnesium oxide (light), magnesium stearate, corn starch, corn swelling starch, mannite, mannitol, mono- and diglyceride of edible fatty acids, montan glycol wax, sodium benzoate, (anhydrous) sodium carbonate, sodium chloride, sodium hydrogen carbonate, poly(butylmethacrylate)-co-(2-dimethyl amino ethyl methacrylate), polyvidone K25, povidone, refined castor oil, sucrose, sucrose monostearate, shellac, sorbitol, talcum, titanium dioxide, tartaric acid. propylene glycol or polyethylene glycol or macrogol, stabilizers, antioxidants, various natural or synthetic emulsifying, dispersing or wetting agents, coloring agents, aromatizing agents, buffers, disintegrating agents, and other substances known in the art to promote the biological availability of the active agent.

A number of studies have demonstrated surprisingly high levels of FAS expression in pre-cancerous human breast lesions, as well as pre-cancerous lesions from other organs. Table 2 below illustrates the prevalence of FAS expression in cancer precursor lesions and their rate of progression to, or association with invasive cancer.

Since the nomenclature of pre-cancerous lesions may differ for each organ, a brief definition of terms for will be helpful to interpret the table. In the breast, there are two varieties of pre-invasive (pre-cancerous) lesions that are defined as in situ carcinoma: intraductal carcinoma and in situ lobular carcinoma (Rows 1 & 2). The term in situ carcinoma is used to describe a lesion in which the pre-cancerous cells have not yet invaded into the surrounding tissue. These lesions are associated with the highest risk for the development of invasive carcinoma and also have the highest prevalence of FAS immunoreactivity. There are also breast lesions of intermediate risk for cancer development (Row 3). These so-called “atypical ductal or lobular hyperplasias” do not exhibit all the histological features of in situ carcinoma. These breast lesions indicate a risk for the development of breast cancer about half that of in situ carcinoma and have a lower frequency of FAS positivity.

In the prostate, prostatic intraepithelial neoplasia (PIN) is a lesion associated with the presence of invasive carcinoma elsewhere in the gland. PIN is described as being low or high grade. Although low grade lesions do not have a significant association with cancer, high-grade PIN occurs with invasive prostate cancer in about a third of cases (Row 4). The true natural history or untreated PIN in yet unknown. FAS is commonly expressed in high grade PIN.

The adenoma is the commonly accepted precursor lesion to colorectal carcinoma (Row 5), as cancer has been shown to commonly arise within or in association with adenomas. Increased size, villous morphology, and the presence of high-grade dysplasia (as defined by both histologic and cytologic features) are associated with an increased risk for the development of cancer. The term “dysplasia” is used to indicate histologic and cytologic changes in tissues that indicate progression to a pre-cancerous lesion. In one study, FAS was ubiquitously present in colorectal adenomas; another group found that FAS expression increased with increasing degrees of dysplasia in the adenomas.

In the lung, squamous carcinoma develops from dysplastic squamous mucosa. Chronic insult to the lung, such as tobacco smoke, leads first to a change from ciliated glandular mucosa in the airways to squamous mucosa which is more resistant to damage. This process is called metaplasia. Over time, the carcinogens in the smoke cause histologic and cytologic changes called dysplasia that indicate the development of a pre-cancerous lesion. Once high grade dysplasia is present, there is a significant risk for the development of invasive cancer. FAS expression has been found to be increased in dysplastic bronchial epithelium.

Cancer precursor lesions in the stomach are adenomas—similar but not identical to colorectal adenomas. As in the colon, they carry an increased risk of cancer development and FAS is commonly expressed.

The precursor to invasive cancer in the oral cavity is dysplasia of the squamous mucosa lining the mouth—similar to bronchial squamous dysplasia that lead to lung cancer. FAS expression is also increased in these dysplastic lesions.

Bile duct cancers arise commonly from dysplastic glandular mucosa. In this tissue, the epithelium does not change from glandular to squamous as in the bronchus. Nonetheless, FAS expression is ubiquitously present in bile duct dysplasia.

TABLE 2
FAS Expression in Cancer Precursor Lesions

| | Pathological | % FAS Positive | Progression to, or Association |
|---|---|---|---|
| Organ | Lesion | Immuno-histochemistry | with Cancer |
| Breast | Intraductal | ˜73% (6) | ˜25% over 16-21.6 yrs. (7-9) |
| | Carcinoma | | |
| Breast | Lobular | 100% (6) | 21.3-36.4% over 15 −> 20 yrs. |
| | Carcinoma | | (10-13) |
| | In Situ | | |
| Breast | Atypical | ˜50% (14) | 5.1-12.9% over 8-21 yrs. |
| | Lobular/Ductal | | (13, 15-17) |
| | Lesions | | |
| Prostate | Prostatic | 96% low grade | ˜33% of men with high grade PIN |
| | Intraepithelial | 100% high grade (18) | have cancer on follow-up biopsy |
| | Neoplasia (PIN) | | (19) |
| Colon | Adenoma | 100% all adenomas (20) | ˜3.7% progress to cancer with |
| | | 4.6%, 17.5%, 56% of | villous or >1 cm adenomas; 0.5% |
| | | adenomas with low, | progress with small tubular |
| | | moderate or high grade | adenomas over 14 yrs. (22) |
| | | dysplasia (21) | |
| Lung | Squamous | Increased FAS expression | 33% of patients with markedly |
| | dysplasia | in all histologically normal | dysplastic cells in sputum |
| | | mucosa and all preneo- | developed lung cancer over |
| | | plastic lesions from | 1-10 yrs. (24, 25) |
| | | patients with squamous | |
| | | carcinoma compared to | |
| | | normal controls (23) | |
| Stomach | Adenoma | 78% positive (26) | 2% over 16 years (27, 28), 11% |
| | | | over 6 mos-12 yrs. (28, 29) |
| Mouth | Squamous | Increased FAS expression | 2.9% annual malignant |
| | dysplasia | in dysplasia compared to | transformation rate median |
| | | normal controls (30) | follow-up of 29 months (31, 32) |
| Bile | Bile duct | 100% of dysplastic lesions | Carcinoma arising in dysplasia |
| Duct | dysplasia | show increased FAS | has been identified in 42% of |
| | | expression (33) | patients (34) |

(6) Milgraum, L. Z., Witters, L. A., Pasternack, G. R., and Kuhajda, F. P., “Enzymes of the fatty acid synthesis pathway are highly expressed in in situ breast carcinoma.” Clin Cancer Res, 3: 2115-2120, 1997.
(7) Bestill, W. L., Rosen, P. P., Lieberman, P. H., and Robbins, G. F., “Intraductal carcinoma. Long-term follow-up after treatment by biopsy alone,” JAMA, 239: 1863-1867, 1978.
(8) Page, D. L., Dupont, W. D., Rogers, L. W., and Landenberger, M., “Intraductal carcinoma of the breast: follow-up after biopsy only,” Cancer, 55: 2698-2708, 1982.
(9) Page, D. L. and Japaze, H. J., The Breast: Comprehensive Management of Benign and Malignant Diseases, p. 169-192. Philadelphia: W. B. Saunders, 1991.
(10) Anderson, J., “Lobular carcinoma in situ: a long-term follow-up in 52 cases,” Acta Pathol Microbiol Scand Sect A, 82: 519-533, 1974.
(11) Rosen, P. P., Lieberman, P. H., Braun, D. W. J., Adair, F., and Braun, D. W. J., “Lobular carcinoma in situ of the breast: detailed analysis of 99 patients with average follow-up of 24 years,” Am J Surg Pathol, 2: 225-251, 1978.
(12) Page, D. L., Kidd, T. E. J., Dupont, W. D., Simpson, J. F., and Rogers, L. W. “Lobular neoplasia of the breast: higher risk for subsequent invasive cancer predicted by more extensive disease,” Hum Pathol, 22: 1232-1239, 1991.
(13) Rosen, P., P. Rosen's breast pathology., 2nd. edition, p. 229-248, 581-626. Philadelphia: Lippincott Williams & Wilkins, 2001.
(15) Bodian, C. A., Perzin, K. H., Lattes, R., Hoffmann, P., and Abernathy, T. G., “Prognostic significance of benign proliferative breast disease,” Cancer, 71: 3896-3907, 1993.
(16) Dupont, W. D. and Page, D. L., “Breast cancer risk associated with proliferative disease, age at first birth, and family history of breast cancer,” Am J Epidemiol, 1225: 769-779, 1987.
(17) Carter, C. L., Corle, D. K., Micozzi, M. S., Schatzkin, A., and Taylor, P. R., “A prospective study of the development of breast cancer in 16,692 women with benign breast disease,” Am J Epidemiol, 128: 467-477, 1988.
(19) Kronz, J. D., Allan, C. H., Shaikh, A. A., and Epstein, J. I., “Predicting cancer following a diagnosis of high-grade prostatic intraepithelial neoplasia on needle biopsy: data on men with more than one follow-up biopsy,” Am J Surg Pathol, 25: 1079-1085, 2001.
(22) Atkin, W. S., Morson, B. C., and Cuzick, J., “Long-term risk of colorectal cancer after excision of rectosigmoid adenomas,” N Engl J Med, 326: 658-662, 1992.
(24) Suprun, H., Hjerpe, A., Nasiell, M., and Vogel, B., Prevention and Detection of Cancer, Part II, Detection., p. 1303-1320, New York: Marcel Dekker, 1980.
(25) Carter, D. and Patchesfsky, A. S. Tumors and tumor-like lesions of the lung., 1st edition, p. 120-147, Philadelphia: W. B. Saunders Co., 1998.
(27) Laxen, F., “Gastric carcinoma and pernicious anemia in long-term endoscopic follow-up of subjects with gastric polyps,” Scand J Gastroenterol, 19: 535-540, 1984.
(28) Goldman, H. Pathology of the gastrointestinal tract, 2 edition, p. 594. Baltimore: Williams and Wilkins, 1998.
(29) Kamiya, T., Morishita, T., Asakura, H., Miura, S., Munakata, Y., and Tsuchiya, M., “Long-term follow-up study on gastric adenoma and its relation to gastric protruded carcinoma,” Cancer, 50: 2496-2503, 1982.
(31) Schepman, K. P., van der Meij, E. H., Smeele, L. E., and van der Waal, I. “Malignant transformation of oral leukoplakia: A follow-up study of a hospital-based population of 166 patients with oral leukoplakia from The Netherlands,” Oral Oncol, 34: 270-275, 1998.
(32) Gnepp, D. R., Diagnostic surgical pathology of the head and neck, 1st edition, p. 1-17. Philadelphia: W. B. Saunders Co., 2000.
(34) Owen, D. A. and Kelly, J., Pathology of the gallbladder, biliary tract, and pancreas., p. 337. Philadelphia: W. B. Saunders Company, 2001

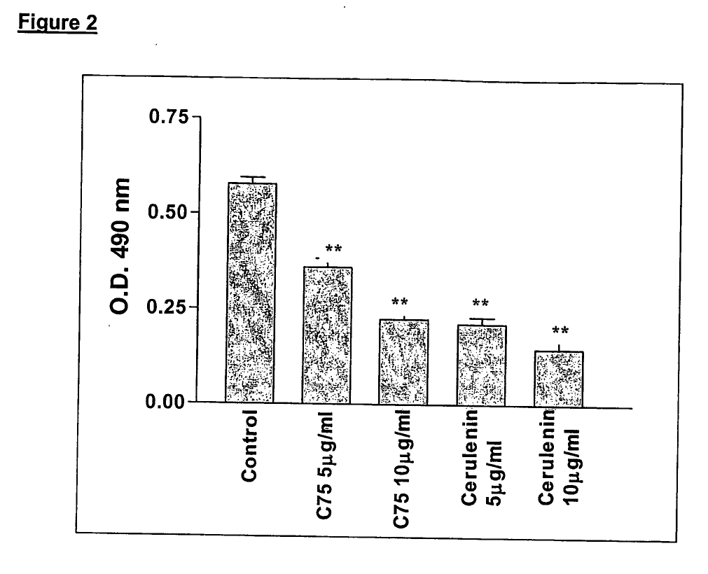

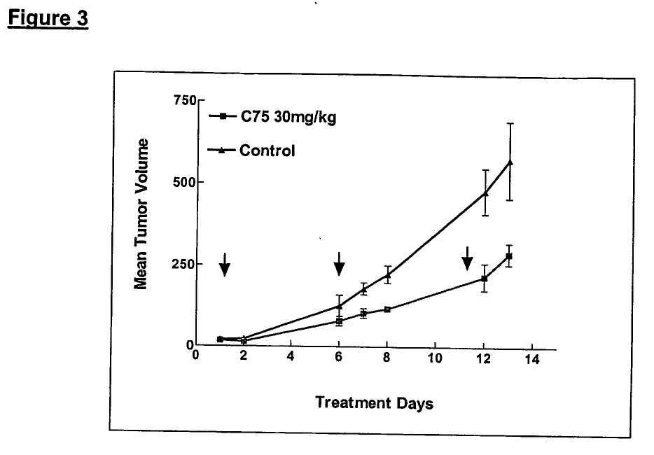

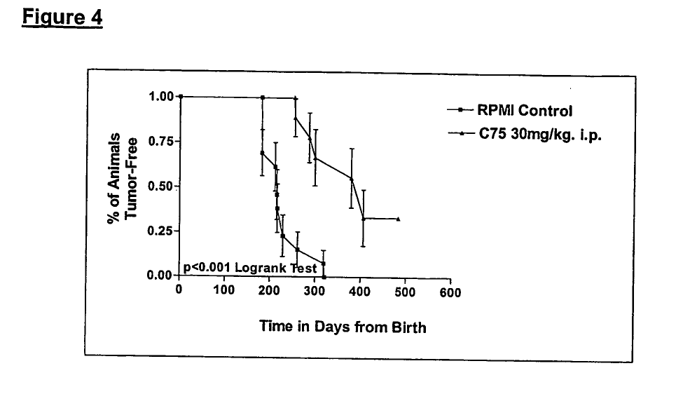

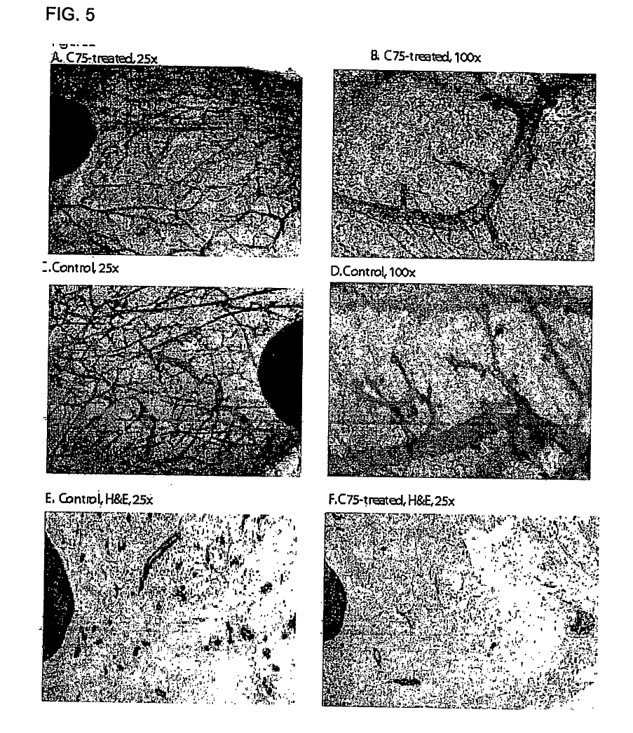

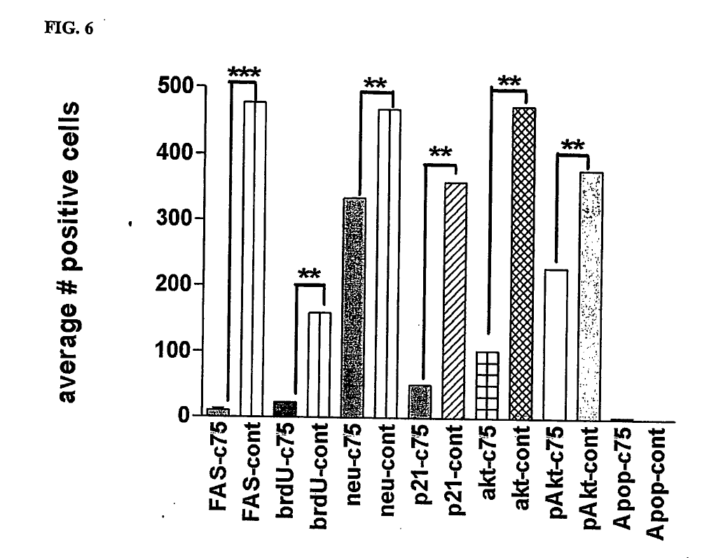

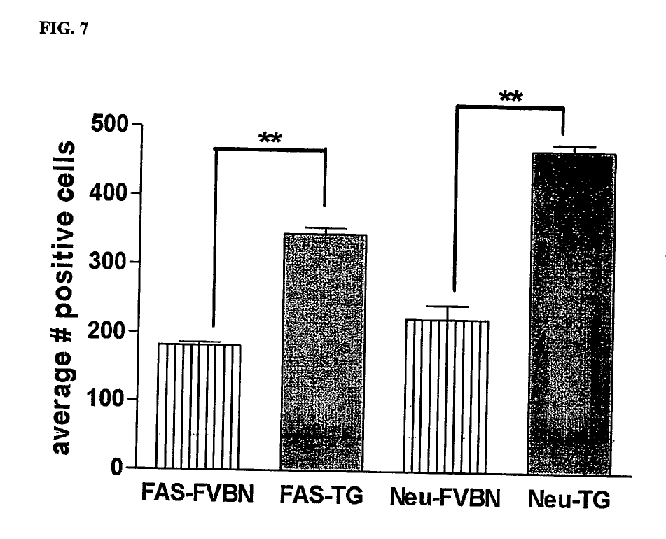

U.S. Pat. No. 5,759,837 discloses that the inhibition of FAS in vitro induces apoptosis in human breast cancer cell lines. This finding is bolstered by Example 2 and FIG. 2 which illustrate the inhibition of NT5 cancer cell growth by the FAS inhibitors cerulenin and C75 in vitro. It is also known that the inhibition of FAS in vivo reduces the growth of human breast and prostate cancer xenografts (Owen, D. A. and Kelly, J., Pathology of the gallbladder, biliary tract, and pancreas., p. 337. Philadelphia: W.B. Saunders Company, 2001; Pizer, E., Pflug, B., Bova, G., Han, W., Udan, M., and Nelson, J., “Increased fatty acid synthase as a therapeutic target in androgen-independent prostate cancer progression.” Prostate, 47: 102-110, 2001). This finding is supported by Example 3 and FIG. 3 which illustrate the reduction in growth of NT5 tumor cell allografts in mice by the FAS inhibitor C75. Thus, it was known that FAS inhibitors can inhibit pre-existing cancer cell growth. However, until now, it was not known that treatment with FAS inhibitors would inhibit cancer development.

To show that FAS inhibitors would inhibit cancer development, the HER-2/neu breast cancer transgenic mouse model was used. Derived from the FVB/N strain, neu-N transgenic mice express the non-transforming rat neu cDNA under the control of a mammary-specific promoter. As a consequence, the mice develop spontaneous mammary adenocarcinomas beginning at approximately 125 days, with nearly all of the mice harboring tumors by 300 days (Guy, C., Webster, M., Schaller, M., Parsons, T., Cardiff, R., and Muller, W., “Expression of the neu protooncogene in the mammary epithelium of transgenic mice induces metastatic disease,” Proc. Natl. Acad. Sci. USA, 89: 10578-10582, 1992). This model does not have an activated (mutated) neu gene. Although the activated neu model has the advantage of more rapid tumor development (Guy, C., Cardiff, R., and Muller, W., “Activated neu induces rapid tumor progression,” Journal of Biological Chemistry, 271: 7673-7678, 1996), this point mutation has not been identified in human breast cancer (Lofts, F. and Gullick, W., “C-erbB2 amplification and overexpression in human tumors,” Cancer Treat. Res., 61: 161-179, 1992). Thus, the HER-2/neu breast cancer transgenic mouse model more closely resembles human disease where neu is overexpressed, not mutated. Moreover, neu is expressed in 25% of human intraductal carcinoma (DCIS) (Glockner, S., Lehmann, U., Wilke, N., Kleeberger, W., Langer, F., and Kriepe, H., “Amplification of growth regulatory genes in intraductal breast cancer is associated with higher nuclear grade but not with progression to invasiveness,” Laboratory Investigation, 81: 565-571, 2001), demonstrating that neu over-expression is an early event in human carcinogenesis, thus further substantiating the neuN model. Since both FAS (Milgraum, L. Z., Witters, L. A., Pasternack, G. R., and Kuhajda, F. P., “Enzymes of the fatty acid synthesis pathway are highly expressed in in situ breast carcinoma”, Clin Cancer Res, 3: 2115-2120, 1997) and neu have been identified in in situ carcinoma in human breast tissues, and inhibition of FAS leads to the apoptosis of breast cancer cells with neu overexpression, the neu-N model was used to show that FAS inhibitors can inhibit cancer development.

As a representative FAS inhibitor, C75 was used. The synthesis and efficacy of C75 as an FAS inhibitor was demonstrated in U.S. Pat. No. 5,981,575.

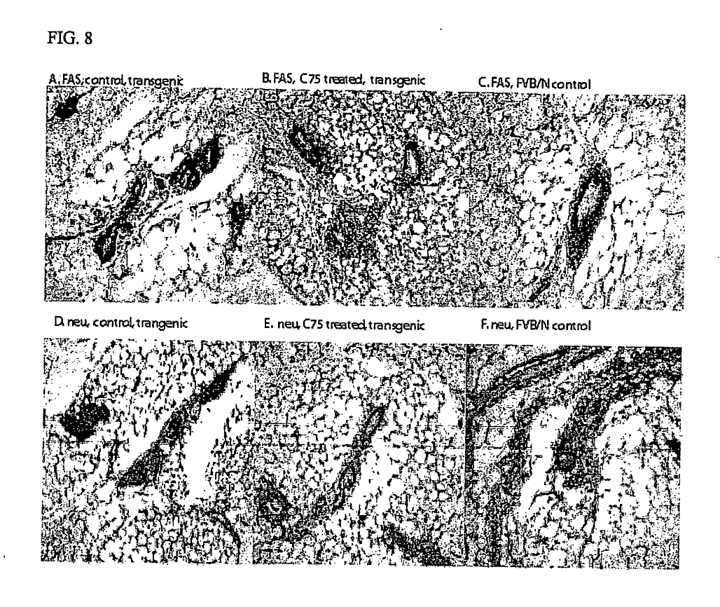

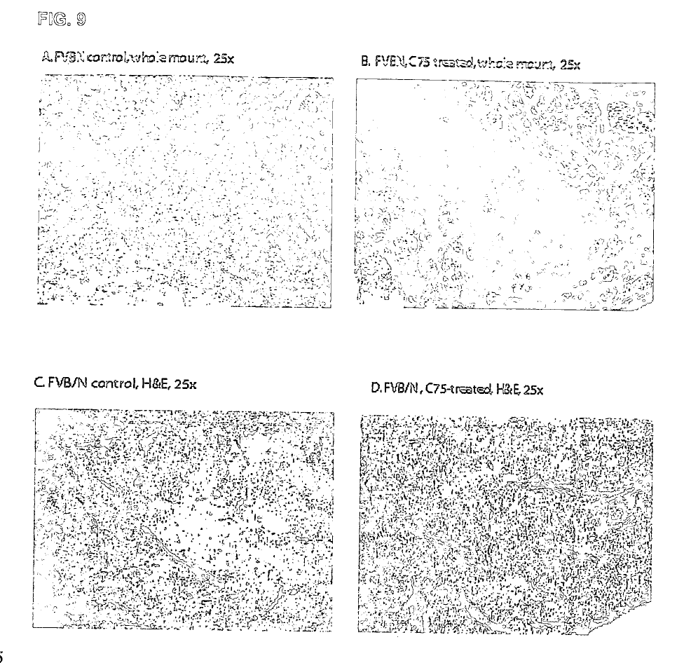

Example 4 and FIG. 4 illustrate that the treatment of HER-2/neu breast cancer transgenic mice with the FAS inhibitor C75 significantly inhibited the development of cancer, with three animals remaining tumor free for nearly 1.5 years, the duration of their lives. Other FAS inhibitors may be expected to function in a manner analogous to C75.

The following examples are provided to further illustrate the methods and compositions of the present invention. These examples are illustrative only and are not intended to limit the scope of the invention in any way.

### EXAMPLE 1

### The Inhibition of Fatty Acid Synthesis by 2,3-epoxy-4-oxo-7,10-dodecadienoylamide (i.e., Cerulenin) and Tetrahydro-3-methylene-2-oxo-5-n-octyl-4-furancarboxylic acid (i.e., C75) in NT5 Cells.

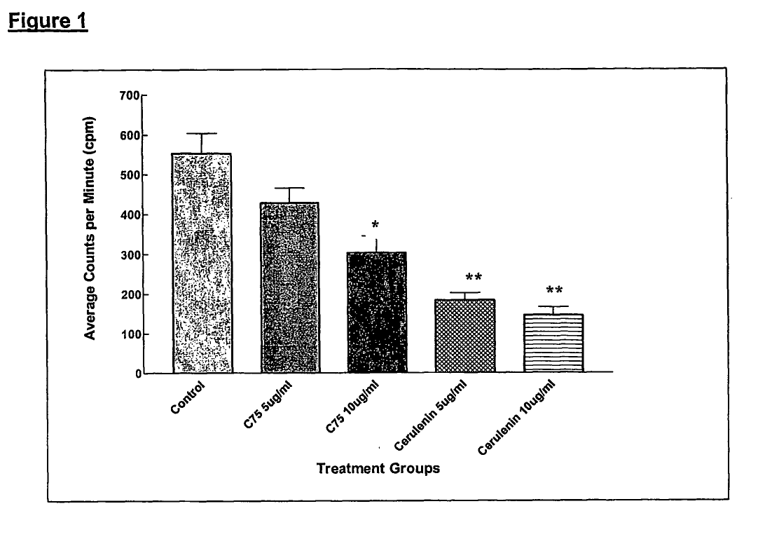

The ability of the FAS inhibitors cerulenin and C75 to inhibit fatty acid synthesis in developing tumors was demonstrated in NT5 cancer cells established from tumors that had developed in transgenic mice. (See FIG. 1). 5×10⁴ NT5 cells were plated in 24-well plates. Following overnight attachment, cells were treated with cerulenin and C75 diluted in DMSO at 5 mg/ml for 4 h, with control cells receiving vehicle alone. During the last 2 h of drug treatment, cells were treated with 1 μCi [¹⁴C]acetate. Total lipids were then extracted and counted. The results are shown in FIG. 1. Statistical analysis (i.e., two tailed t-tests) of the results are as follows: Control-C75 5 μg/ml, p=0.116; Control-C75 10 μg/ml, p=0.018; Control-Cerulenin 5 μg/ml, p=0.002; Control-Cerulenin 10 μg/ml, p=0.002.

FIG. 1 shows the inhibition of fatty acid synthesis by cerulenin and C75 in NT5 cancer cells. NT cell lines are established from tumors that developed in transgenic mice (Reilly, R., Gottlieb, M., Ercolini, A., Machiels, J., Kane, C., Okoye, F., Muller, W., Dixon, K., and Jaffee, E., “HER-2neu Is a Tumor Rejection Target in Tolerized HER-2/neu Transgenic Mice,” Cancer Research, 60: 3569-3576, 2000; Reilly, R., Machiels, J., Emens, L., Ercolini, A., Okoye, F., Lei, R., Weintraub, D., and Jaffee, E., “The Collaboration of Both Humoral and Cellular HER-2/neu-targeted Immune Responses Is Required for the Complete Eradication of HER-2/neu-expressing Tumors,” Cancer Research, 61: 880-883, 2001), and provide an in vitro model for testing the FAS inhibitors C75 and cerulenin. As can be seen, both cerulenin and C75 inhibit fatty acid synthesis in NT5 cells at levels similar to previous studies with human cell lines (Pizer, E. S., Thupari, J., Han, W. F., Pinn, M. L., Chrest, F. J., Frehywot, G. L., Townsend, C. A., and Kuhajda, F. P., “Malonyl-coenzyme-A is a potential mediator of cytotoxicity induced by fatty acid synthase inhibition in human breast cancer cells and xenografts,” Cancer Research, 60: 213-218, 2000; Pizer, E., Pflug, B., Bova, G., Han, W., Udan, M., and Nelson, J., “Increased fatty acid synthase as a therapeutic target in androgen-independent prostate cancer progression,” Prostate, 47:102-110, 2001). Moreover, FIG. 1 also demonstrates that these cells have active fatty acid synthesis, thus expressing FAS, the target enzyme for these inhibitors.

### EXAMPLE 2

### The Inhibition of NT5 Cancer Cell Growth In Vitro by FAS Inhibitors

The ability of FAS inhibitors to inhibit the growth of NT5 cancer cells was demonstrated in vitro. (See FIG. 2). 1×10⁴ cells were plated in 24-well plates. Following overnight attachment, cells were treated with C75 or cerulenin diluted in DMSO at 5 mg/ml, with control cells receiving vehicle alone. After 72 hours, cells were stained with crystal violet (0.2% in 10% methanol), solubilized in 1% SDS, and the O.D. measured at 490 nm. Two-tailed t-test: Control-C75 5 μg/ml, p=0.0003; Control-C75 10 μg/ml, p<0.0001; Control-Cerulenin 5 μg/ml, p<0.0001; Control-Cerulenin 10 μg/ml, p<0.0001.

FIG. 2 shows the inhibition of NT5 cancer cell growth by FAS inhibitors in vitro. As can be seen, treatment with the FAS inhibitors, cerulenin and C75 significantly reduced the growth of the cancer cells (as indicated by the reduced O.D. 490 nm).

### EXAMPLE 3

### The Reduction in the Growth of NT5 Cancer Cell Allografts in Mice by FAS Inhibitors

The ability of FAS inhibitors to inhibit the growth of NT5 cancer cell allografts in mice was demonstrated using FVB/N mice. (See FIG. 3). Fourteen animals received 0.1 ml packed cultured NT5 cells in the flank. When measurable tumors appeared, seven animals were treated with C75 (30 mg/kg in 0.1 ml RPMI, intraperitoneal injection) every six days and seven animals received vehicle control. Error bars in FIG. 3 represent standard error of the mean.

FIG. 3 shows the reduction in the growth of NT5 cancer cell allografts in mice by the FAS inhibitor, C75. As can be seen, treatment with C75 significantly reduced the growth of NT5 tumor cell allografts in FVB/N mice.

### EXAMPLE 4

### The Inhibition of Cancer Development by FAS Inhibitors

The ability of FAS inhibitors to inhibit cancer development was demonstrated using the HER-2/neu breast cancer transgenic mouse model. (See FIG. 4) Thirty HER-2/neu breast cancer transgenic mice were used for the study. Fifteen (15) mice received weekly doses of C75 (30 mg/kg in 0.1 ml RPMI) for three months beginning at 5 weeks of age and 15 mice received vehicle alone. Mice were observed daily and the first appearance of breast tumors were recorded. Two (2) mice in the controls and 6 in the treated group died during the study. Log-rank analysis of the data shows that tumor development in the C75 treated animals was significantly delayed. Fifty-percent (50%) of the control mice developed tumors after approximately 200 days versus 300 days for the C75 treated animals. Moreover, three treated animals remained tumor free for nearly 18 months, the duration of their lives.

### EXAMPLE 5

### Investigation into Mechanism of Action

Fifteen 8-10 week old, neu-N transgenic mice were treated intraperitoneally (ip) with C75 at 30 mg/kg weekly, along with fifteen vehicle controls (RPMI). Three mice from the treatment and control groups were sacrificed by carbon dioxide asphyxiation at two-week intervals beginning at week two (two weeks after the first C75 treatment at 8-10 weeks of age). All animals were injected with 1 mg of BrdU two hours prior to sacrifice. Entire inguinal mammary glands were removed along with the intramammary lymph node that was grossly identifiable. Additionally, kidneys, liver and skin samples were collected from each animal. The mammary liver from one side and the kidneys, liver and skin samples were fixed in neutral-buffered formalin, the other was fixed in Carnoy's fixative for whole-mount preparation. In addition, mammary glands from a non-transgenic age-matched FVB/N control mouse were removed for similar analysis at week 10 (age 18-20 weeks).

Following fixation in 10% neutral-buffered formalin for 24 hours, the mammary glands were embedded in paraffin. Six 4 micron slides were prepared from each tissue block, with the first slide stained with hematoxylin and eosin. The remaining unstained sections were utilized for immunohistochemical analysis of the preneoplastic lesions and surrounding breast tissue with the following antibodies: FAS, BrdU and p21/Waf-1 (Dako, Carpinteria, Calif.), Akt and Phospho-Akt (Cell Signaling Technology, Beverly, Mass.), and neu (Santa Cruz Biotechnology, Inc., Santa Cruz, Calif.) apoptosis (ApopTag Peroxidase In Situ Oligo Ligation Kit, Serologicals Corporation, Temecula, Calif.). Staining was assessed by counting the number of positive cells per 500 total cells in the ductal and lobular structures at 400×. Statistical analysis was performed using t-tests on Prism 3 software. The Carnoy's fixed tissue was stained with carmine red as described and whole-mounted on glass slides.

Following 8-10 weeks of C75 treatment, there was a significant reduction of both the number of mammary duct structures, their thickness and the number of budding epithelial structures in the neu-N animals compared to vehicle controls and FVB/N animals.

FIG. 5 shows abnormal mammary gland development in N-neu transgenic mice treated with C75 (pictures A, B, and F) versus controls (pictures C,D, and E.) Picture A shows a whole mount specimen of C75-treated animal which exhibits a significant reduction in the number and caliber of ducts, as well as a decreased number of epithelial structures. An enlarged version of this is shown in Picture B. Pictures A and B may be compared to Pictures C and D respectively, which show a control specimen having normal number, caliber, and budding of duct structures. These changes are reflected in histologic sections in Pictures E and F. Black arrows in A, C, E, and F denote lymph nodes, indicating similar image capture areas in both specimen types.

As shown in FIGS. 6 and 6, apoptotic changes were increased, DNA synthesis was decreased, and FAS, neu, Akt, Phospho-Akt and p21/Waf1 expression were all decreased when compared to controls and FVB/N mice. FIG. 8 shows immunohistolochemical staining for FAS and neu(hematoxylin counterstain) in C75 treated neu-N transgenic mice and vehicle controls in FVB/N control mice. In vehicle control animals, high levels of FAS expression were present in both ducts and adipose tissue with strong diffuse staining (Picture A) (All pictures in FIG. 5 are 200× magnification). C75 treated animals had significantly lower FAS expression in both breast ducts and adipose tissue with weak and focal staining (Picture B). FAS expression in the FVB/N control animals was rare and weak (Picture C). Immunohistochemical staining from neu was decreased in the C75 animals (Picture E) compared to vehicle control animals (Picture D). In FVB/N control animals, neu expression was focal and weak (Picture F).

Importantly, these effects were restricted to the breast epithelial cells that overexpress neu, and not to other normal duct structures in the skin, liver or kidney. In the FVB/N animals there was no significant morphological difference in mammary structures between the C75 treated animals and the controls. This can be seen in FIG. 9, which shows normal mammary gland development in FVB/N control mice treated with C75 (Pictures B and D) versus controls (Pictures A and C). No significant morphological differences in mammary structures are apparent.

We claim:

1. A method of inhibiting cancer development comprising the administration to a subject in need thereof of an effective amount of a fatty acid synthase inhibitor.

2. A method according to claim 1 wherein the subject is a mammal.

3. A method according to claim 1 wherein the subject is a human.

4. A method according to claim 1 wherein the subject has pre-cancerous lesions.

5. A method according to claim 5 wherein the pre-cancerous lesions express fatty acid synthase.

6. A method according to claim 5 wherein the pre-cancerous lesions express the neu protein.

7. A method according to claim 5 wherein the pre-cancerous lesions express fatty acid synthase and the neu protein.

8. A method according to claim 5 wherein the pre-cancerous lesions are in a tissue type selected from the group consisting of breast, prostate, colon, lung, stomach, mouth, and bile duct.

9. A method according to claim 8 wherein the tissue type is breast.

10. A method according to claim 8 wherein the tissue type is prostate.

11. A method according to claim 8 wherein the tissue type is colon.

12. A method according to claim 8 wherein the tissue type is lung.

13. A method according to claim 8 wherein the tissue type is stomach.

14. A method according to claim 8 wherein the tissue type is mouth.

15. A method according to claim 8 wherein the tissue type is bile duct.

16. A method according to claim 1 wherein the effective amount is in the range from about 60 mg/kg to about 7.5 mg/kg per day.

17. A method according to claim 1 wherein the fatty acid synthase inhibitor is a compound that directly inhibits the fatty acid synthase enzyme.

18. A method according to claim 1 wherein the fatty acid synthase inhibitor is a compound having the following formula: 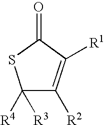
wherein:
R¹═H, C₁-C₂₀ alkyl, cycloalkyl, alkenyl, aryl, arylalkyl, or alkylaryl, —CH₂OR⁵, —C(O)R⁵, —CO(O)R⁵, —C(O)NR⁵R⁶, —CH₂C(O)R⁵, or —CH₂C(O)NHR⁵, where R⁵ and R⁶ are each independently H, C₁-C₁₀ alkyl, cycloalkyl, alkenyl, aryl, arylalkyl, or alkylaryl, optionally containing one or more halogen atoms.
R²═—OH, —OR⁷, —OCH₂C(O)R⁷, —OCH₂C(O)NHR⁷, —OC(O)R⁷, —OC(O)OR⁷, —OC(O)NR⁷R⁸, where R⁷ and R⁸ are each independently H, C₁-C₂₀ alky, cycloalkyl, alkenyl, aryl, arylalkyl, or alkylaryl, and where R⁷ and R⁸ can each optionally contain halogen atoms;
R³ and R⁴, the same or different from each other, are C₁-C₂₀ alkyl, cycloalkyl, alkenyl, aryl, arylalkyl, or alkylaryl.

19. A method according to claim 1 wherein the fatty acid synthase inhibitor is a compound having the following formula: 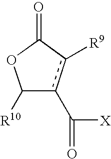
R⁹═H, or C₁-C₂₀ alkyl, cycloalkyl, alkenyl, aryl, arylalkyl, or alkylaryl, ═CHR¹¹, —C(O)OR¹¹, —C(O)R¹¹, —CH₂C(O)OR¹¹, —CH₂C(O)NHR¹¹, where R¹¹ is H or C₁-C₁₀ alkyl, cycloalkyl, or alkenyl;
R¹⁰═C₁-C₂₀ alkyl, cycloalkyl, alkenyl, aryl, arylalkyl, or alkylaryl;
X═—OR¹², or —NHR¹², where R¹² is H, C₁-C₂₀ alkyl, cycloalkyl, alkenyl, aryl, arylalkyl, or alkylaryl, the R¹² group optionally containing a carbonyl group, a carboxyl group, a carboxyamide group, an alcohol group, or an ether group, the R¹² group further optionally containing one or more halogen atoms;
with the proviso that when R⁹ is ═CH₂, then X is not —OH.

20. A method according to claim 1 wherein the fatty acid synthase inhibitor is tetrahydro-3-methylene-2-oxo-5-n-octyl-4-furancarboxylic acid.
